(19) United States
(12) Plant Patent Application Publication
(10) Pub. No.: US 2016/0360676 P1
(43) Pub. Date: Dec. 8, 2016

(54) Pelargonium plant named 'KLEPZ15403'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/998,912

(22) Filed: Mar. 2, 2016

(30) Foreign Application Priority Data
Jun. 3, 2015 (CH) 15-3015

(51) Int. Cl. A01H 5/00

(52) U.S. Cl. PLT/329

(57) ABSTRACT

A new pelargonium plant particularly distinguished by pink purple flowers with a purple red eye, and an upright growth habit, is disclosed.

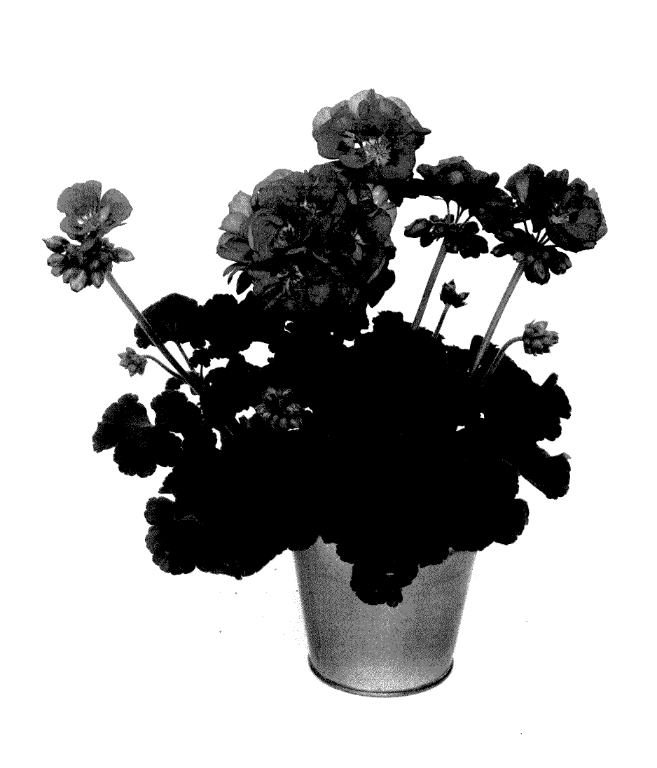

### CROSS-REFERENCE TO RELATED APPLICATIONS

This application claims priority to and the benefit of Switzerland Plant Breeders Rights application filed on Jun. 3, 2015, Application No. 15-3015, the entire contents of which are incorporated herein by reference for all purposes.

An application with the European Community Plant Variety Office was filed on Jul. 1, 2015, File No. 2015-1526.

### GENUS AND SPECIES

Pelargonium zonale

### VARIETY DENOMINATION

‘KLEPZ15403’

### BACKGROUND OF THE NEW PLANT

The present invention comprises a new and distinct variety of pelargonium, botanically known as Pelargonium zonale and hereinafter referred to by the variety name ‘KLEPZ15403’. ‘KLEPZ15403’ originated from a cross-combination in June 2010 in Stuttgart, Germany between the female pelargonium variety ‘PZ-2006-0158’ (unpatented) and the proprietary male pelargonium variety ‘PZ-2007-0004’’ (unpatented).

Seeds from the cross-combination in June 2010 in Stuttgart, Germany were obtained. The seeds were sown and plants were grown for evaluation. A single plant was selected in June 2011 and subsequently named ‘KLEPZ15403’. In September 2011 ‘KLEPZ15403’ was first vegetatively propagated in Stuttgart, Germany by vegetative cuttings from stems. ‘KLEPZ15403’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.
1. Pink purple flowers with a purple red eye; and
2. An upright growth habit.

### DESCRIPTION OF THE PHOTOGRAPH

This new pelargonium plant is illustrated by the accompanying photograph which shows an overall view of the plant. The photograph is of an approximately ten-week-old plant grown in Stuttgart, Germany under greenhouse conditions in May 2014. The colors shown are as true as can be reasonably obtained by conventional photographic procedures.

### DESCRIPTION OF THE NEW VARIETY

The following detailed descriptions set forth the distinctive characteristics of ‘KLEPZ15403’. The data which define these characteristics were collected from asexual reproductions carried out in Stuttgart, Germany. Data was collected on plants grown 2 and a half-months after planting in Stuttgart, Germany under greenhouse conditions in June 2015. Color references are to The R.H.S. Colour Chart of The Royal Horticultural Society of London (R.H.S.), 5^{th} edition (2007).
Classification:
   Family.—Geraniaceae.
   Botanical.—Pelargonium zonale.
   Common.—Pelargonium.
   Designation.—‘KLEPZ15403’.
Parentage:
   Female parent.—The female pelargonium variety ‘PZ-2006-0158’ (unpatented).
   Male parent.—The proprietary male pelargonium variety ‘PZ-2007-0004’’ (unpatented).
Plant:
   Form.—Bushy and round.
   Habit.—Upright.
   Height.—15.0 cm to 18.0 cm.
   Spread.—21.0 cm to 24.0 cm.
   Natural flowering season.—Spring to autumn.
   Propagation type.—Vegetative cuttings.
   Time to produce a finished flowering plant.—9 to 10 weeks.
   Time to initiate and develop roots.—18 days.
   Root description.—White and fine, RHS 155 D.
Stems:
   Quantity of stems per plant.—4 to 5.
   Color.—RHS 137B and RHS 176D.
   Surface texture.—Sparsely pubescent.
   Stem form.—Round.
   Diameter.—0.8 cm to 1.0 cm.
   Length.—9.0 cm to 12.0 cm.
   Internode length.—1.0 cm to 3.0 cm.
   Anthocyanin.—Present, color is RHS 177B.
Leaves:
   Arrangement.—Alternate.
   Shape.—Kidney shaped.
   Apex.—Obtuse, round.
   Base.—Open.
   Margin.—Crenate.
   Surface texture.—Upper surface: Rough, sparsely pubescent Lower surface: Slightly rough to smooth, sparsely pubescent.
   Length.—5.0 cm to 7.0 cm.
   Width.—6.0 cm to 9.0 cm.
   Color.—Upper surface: RHS 137A Lower surface: RHS 137C.
   Petiole.—Length: 4.0 cm to 7.0 cm Diameter: 0.4 cm Color: RHS 137B Strength: Medium.
   Venation pattern.—Palmate.
   Venation color.—Upper surface: RHS 137B Lower surface: RHS 137D.
Inflorescence:
   Lastingness of flowers on the plant.—5 days.
   Umbels.—Quantity of umbels per plant: 4 Length: 4.0 cm to 5.0 cm Diameter: 7.0 cm to 9.0 cm.
   Individual florets of the umbel.—Quantity of florets per umbel: 8 to 10 Depth: 2.5 cm Diameter: 4.0 cm.
   Fragrance.—Absent.
Flower buds:
   Cluster shape.—Round.
   Cluster size.—Length: 3.5 cm Width: 4.0 cm.
   Surface texture.—Moderately pubescent.
   Bud.—Height: 1.0 cm to 1.5 cm Width: 0.7 cm Shape: Elliptic Apex: Peaked Base: Blunt Margin: Entire Color: RHS 137C.
Petals:
   Quantity per flower.—6 to 7 and petaloids.
   Shape.—Drop-shaped.
   Apex.—Round.
   Base.—Attenuate.
   Margin.—Entire.
   Length.—2.5 cm.
   Width.—1.6 cm.
   Surface texture.—Upper surface: Smooth Lower surface: Smooth.
   Color of lobes.—Upper lobes: Upper surface: RHS N66A, RHS 61B, and RHS 69C Lower surface: RHS N74A Lower lobes: Upper surface: RHS N66A, RHS 61B, and RHS 69C Lower surface: RHS N74A.
Petaloids:
   Quantity per flower.—2 to 5.
   Shape.—Moderate.
   Apex.—Round.
   Base.—Attenuate.
   Margin.—Blunt.
   Texture (upper and lower surfaces).—Smooth.
   Length.—0.3 cm to 1.0 cm.
   Width.—0.1 cm to 0.6 cm.
   Color.—Upper surface: RHS N66A and RHS 69C Lower surface: RHS N74A.
Sepals:
   Number per flower.—5.
   Shape.—Elliptic.
   Apex.—Peaked.
   Base.—Obtuse.
   Margin.—Entire.
   Surface texture.—Upper surface: Smooth Lower surface: Moderately pubescent.
   Color.—Upper surface: RHS 137D Lower surface: RHS 137D.
   Length.—1.1 cm.
   Width.—0.3 cm.
Pedicel:
   Length.—1.0 cm to 2.0 cm.
   Diameter.—0.15 cm.
   Color.—RHS 137D and RHS 174B.
   Surface texture.—Moderately pubescent.
   Strength.—Medium.
Peduncle:
   Length.—10.0 cm to 13.0 cm.
   Diameter.—0.5 cm.
   Color.—RHS 137C and RHS 174B.
   Surface texture.—Rough, moderately pubescent.
   Strength.—Strong to medium.
Reproductive organs:
   Stamens.—Quantity of stamens per flower: 1 to 5 (maximum) Anther length: 0.15 cm Filament length: 0.8 cm Filament color: RHS 49C Pollen amount: Moderate Pollen color: RHS N25A
   Pistils.—Number: 1 Stigma color: RHS 60C Style length: 0.5 cm Style color: RHS 60B Ovary arrangement: Central Ovary color: RHS 145D Ovary diameter: 0.2 cm Ovary length: 0.4 cm.
Fruit and seed set: None observed
Disease and pest/insect resistance: None observed

### COMPARISON WITH PARENTAL LINES AND KNOWN VARIETY

‘KLEPZ15403’ is distinguished from its parents as described in Table 1:

TABLE 1
Comparison with Parental Lines

| | | Female parent | Male Parent |
|---|---|---|---|
| Characteristic | ‘KLEPZ15403’ | ‘PZ-2006-0158’ | ‘PZ-2007-0004” |
| Flower color | Pink purple with | Pink with a purple | Purple with an |
| | a purple red eye | eye | orange center |
| Zone on the leaves | Very weak | Absent | Strong |

‘KLEPZ15403’ can be compared to the commercial variety SUNRISE ‘Evita’, also known as ‘KLEPZ11276’ (U.S. Plant Pat. No. 24,429). Differences between the two varieties are described in Table 2:

TABLE 2
Comparison with Similar Variety

| | | Commercial line |
|---|---|---|
| Characteristic | ‘KLEPZ15403’ | SUNRISE ‘Evita’ |
| Flower color | Pink purple with a | Purple with an orange center |
| | purple red eye | |
| Zone on the leaves | Very weak | Strong |

We claim:

1. A new and distinct variety of pelargonium plant named ‘KLEPZ15403’ as illustrated and described herein.
